(19) United States
(12) Patent Application Publication
(10) Pub. No.: US 2019/0031595 A1
(43) Pub. Date: Jan. 31, 2019

(54) COPPER CONTAINING CATALYST FOR PREPARATION OF ALIPHATIC AMINES

(71) Applicant: RHODIA OPERATIONS, Paris (FR)

(72) Inventors: Shengyin YE, Shanghai (CN); Javier DIAZ-MAROTO CARPINTERO, Shanghai (CN)

(73) Assignee: RHODIA OPERATIONS, Paris (FR)

(21) Appl. No.: 16/074,548

(22) PCT Filed: Feb. 4, 2016
(86) PCT No.: PCT/CN2016/073488

§ 371 Date: Aug. 1, 2018

(51) Int. Cl. C07C 209/16; B01J 23/72; B01J 21/08

(52) U.S. Cl. CPC C07C 209/16; C07C 211/08; B01J 21/08; B01J 23/72

(57) ABSTRACT

Provided is a process of reacting an aliphatic alcohol with an aminating agent for obtaining an aliphatic amine, wherein the reaction is carried out in a catalyst comprising from 68 wt % to 100 wt % of a copper oxide and from 0 wt % to 0.1 wt % of a metal co-catalyst, and optionally from 0 wt % to 32 wt % of a catalyst support, weight percentage is based on the total weight of the catalyst.

### TECHNICAL FIELD

The present invention relates to a process for preparation of aliphatic amines which comprises the step of reacting an aliphatic alcohol with an aminating agent in the presence of a copper containing catalyst.

### BACKGROUND ART

Aliphatic amines are of considerable industrial importance and find application in almost every filed of modern technology, agriculture and medicine. Aliphatic amines, such as tertiary amines, can be used for as intermediates for disinfectants, foam boosters for household liquid detergents, active agents for hair conditioners, softeners for clothes, reagents for mild dyeing, etc.

Among the various known processes for the preparation of aliphatic amines, one process is one-step amination of aliphatic alcohols, such as long chain fatty alcohols, with various starting amines, such as ammonia, primary and secondary amines. For example, the process may be a reaction of an aliphatic alcohol with a dimethylamine to yield the corresponding alkyldimethylamine. Such reaction is initiated by the dehydrogenation of a starting aliphatic alcohol to the corresponding aldehyde, with the generation of two hydrogens, as shown in the reaction scheme below:

RCH₂OH→RCHO+2H (1) dehydrogenation of a starting aliphatic alcohol

RCHO+Me₂NH→RCH(OH)NMe₂ (2) non-catalytic addition of Me₂NH to an aldehyde

RCH(OH)NMe₂+2H→RCH₂NMe₂+H₂O (3) hydrogenolysis of the adduct to a tertiary amine

RCH(OH)NMe₂→R′CH═CHNMe₂+H₂O (4) dehydration of the adduct to form an enamine

R′CH═CHNMe₂+2H→RCH₂NMe₂ (5) hydrogenation of an enamine to a tertiary amine

The addition of Me₂NH to the generated aldehyde proceeds non-catalytically to form the corresponding aldehyde-amine adduct, followed by hydrogenolysis of the adduct to the final tertiary amine RCH₂NMe₂, with liberation of water or by dehydration of the adduct to form an enamine, which is then hydrogenated to the final RNMe₂. Amination of the aliphatic alcohol with a primary amine such as MeNH₂ proceeds by the same reaction mechanism to form first the corresponding secondary amine, RNHMe, which reacts again with the starting aliphatic alcohol to form the dialkyl tertiary amine, R₂NMe. Amination with ammonia proceeds by a similar stepwise mechanism to form trialkyl amines R₃N, via the formation of intermediate RNH₂ and R₂NH. The reaction scheme shown above suggests that supply of bulk hydrogen is not necessary for the hydrogenolysis step (3) and hydrogenation step (5) because the required hydrogen is generated by the dehydrogenation of the starting aliphatic alcohol. However, the process is preferably carried out in the presence of additional hydrogen gas.

Various catalysts have been studied for the preparation of alkylamines. For example, U.S. Pat. No. 4,293,716 discloses a process for preparing alkyldimethylamines which comprises passing through a fixed bed containing 22 wt % of CuO. The catalyst bed is composed of a suitable support, such as silica gel and alumina. U.S. Pat. No. 4,409,399 discloses a process for producing aliphatic amines which comprises reacting an aliphatic alcohol with an aminating agent, such as a secondary amine, in the presence of an unsupported catalyst which may consist of a copper oxide and a nickel oxide.

For the preparation of the aliphatic amines by using the above mentioned process, it is desired to obtain high conversion rate of the aliphatic alcohols, at the same time, to maintain minimal level of side reactions. For example, in the preparation of alkyldimethylamines by reacting an aliphatic alcohol with a dimethylamine, one significant side reaction is the disproportionation of Me₂NH to MeNH₂ and Me₃N, which will decrease the yield of the target tertiary amines. It remains a challenge to provide a catalyst which can lead to high efficiency and good selectivity of the process.

### SUMMARY OF INVENTION

It has been surprising found that the above objective can be achieved by the present invention.

In one aspect of the present invention, there is provided a process of reacting an aliphatic alcohol of formula (I)

R₁CH₂OH (I),

wherein R₁ is a linear or branched, saturated or unsaturated aliphatic group having from 3 to 21 carbon atoms, with an aminating agent of formula (II)

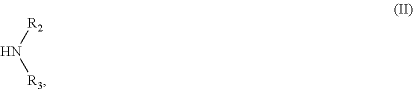

wherein R₂ and R₃, the same or different, are hydrogen or a linear or branched, saturated or unsaturated aliphatic group having from 1 to 24 carbon atoms, for obtaining an aliphatic amine of formula (III), (IV) or (V)

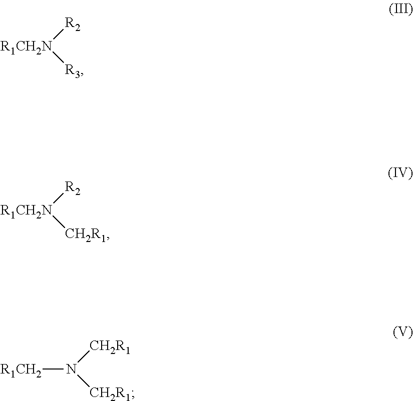

wherein the reaction is carried out in the presence of a catalyst comprising from 68 wt % to 100 wt % of a copper oxide and from 0 wt % to 0.1 wt % of a metal co-catalyst; and optionally from 0 wt % to 32 wt % of a catalyst support, weight percentage is based on the total weight of the catalyst.

Preferably, the catalyst comprises from 75 wt % to 100 wt % of a copper oxide and from 0 wt % to 0.1 wt % of a metal co-catalyst, and optionally from 0 wt % to 25 wt % of a catalyst support, weight percentage is based on the total weight of the catalyst.

Notably, the catalyst comprises from 68 wt % to 95 wt % of a copper oxide as the sole catalytic metal, and optionally from 5 wt % to 32 wt % of a catalyst support, weight percentage is based on the total weight of the catalyst.

In particular, the catalyst comprises from 75 wt % to 100 wt % of a copper oxide as the sole catalytic metal, and optionally from 0 wt % to 25 wt % of a catalyst support, weight percentage is based on the total weight of the catalyst.

In some aspects, the catalyst consists of from 68 wt % to 95 wt % of a copper oxide and from 5 wt % to 32 wt % of a catalyst support, weight percentage is based on the total weight of the catalyst.

Notably, the catalyst consists of from 75 wt % to 95 wt % of a copper oxide and from 5 wt % to 25 wt % of a catalyst support, weight percentage is based on the total weight of the catalyst.

The catalyst support may be silica.

The aminating agent may have the formula (VI)

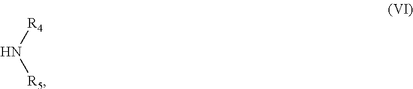

wherein R₄ and R₅, the same or different, are a linear or branched, saturated or unsaturated aliphatic group having from 1 to 24 carbon atoms, the aliphatic amine has the formula (VII):

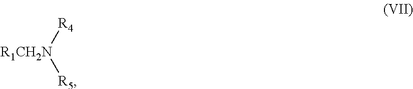

wherein R₁ is a linear or branched, saturated or unsaturated aliphatic group having from 3 to 21 carbon atoms, R₄ and R₅ are as defined in formula (VI).

Preferably, the aliphatic alcohol and the aminating agent are mixed together with a flow of hydrogen and the mixture is continuously introduced into a reaction zone, wherein the molar ratio of the aliphatic alcohol/the aminating agent/the hydrogen is in the range of from 1:1:5 to 1:2:20.

More preferably, the molar ratio of the aliphatic alcohol/the aminating agent/the hydrogen is in the range of from 1:1:5 to 1:1.2:15.

The reaction may be carried out at a temperature of from 150° C. to 350° C.

Preferably, the reaction is carried out at a temperature of from 200° C. to 250° C.

The reaction may be carried out under a pressure of from 0 to 5 barg.

Preferably, the reaction is carried out under a pressure of from 0 to 0.5 barg.

### DETAILED DESCRIPTION

Throughout the description, including the claims, the term “comprising one” or “comprising a” should be understood as being synonymous with the term “comprising at least one”, unless otherwise specified, “between” and “from . . . to . . . ” should be understood as being inclusive of the limits.

As used herein, “weight percent,” “wt %,” “percent by weight,” “% by weight,” and variations thereof refer to the concentration of a substance as the weight of that substance divided by the total weight of the composition and multiplied by 100.

The present invention relates to a process for preparation of aliphatic amines by reacting an aliphatic alcohol with an aminating agent selected from ammonia, primary amines, secondary amines and a mixture thereof. The aliphatic alcohol of the process has the formula (I):

R₁CH₂OH (I)

wherein R₁ is a linear or branched, saturated or unsaturated aliphatic group having from 3 to 21 carbon atoms, preferably from 3 to 17 carbon atoms, more preferably, from 7 to 17 carbon atoms.

The aminating agent of the invention may be selected from the group consisting of ammonia, primary amines, secondary amines and a mixture thereof. It is appreciated that the aminating agent may be a single species of amine compound or a mixture of more than one amine compounds. The aminating agent of the process is represented by the formula (II):

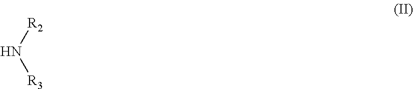

wherein R₂ and R₃, the same or different, are hydrogen or a linear or branched, saturated or unsaturated aliphatic group having from 1 to 24 carbon atoms, preferably from 1 to 18 carbon atoms, more preferably, from 1 to 4 carbon atoms.

The aliphatic amines that are formed herein can be represented by the formula (III), (IV) or (V):

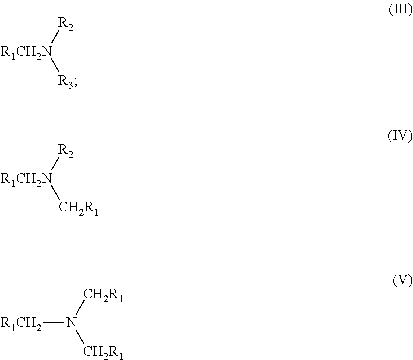

wherein R₁, R₂ and R₃ are as defined above.

Examples of aliphatic alcohols that can be used herein include 1-octanol, 1-decanol, 1-dodecanol, 1-tetradecanol, 1-hexadecanol, 1-octadecanol, 2-ethyl-1-hexanol, oleyl alcohol, 1-nonanol and mixtures thereof.

Primary amines that can be used herein include monomethylamine, monoethylamine, dodecylamine, hexadecylamine, 2-ethylhexylamine and mixtures thereof. Secondary amines that can be used herein include dimethylamine, diethylamine, dodecylmethylamine, dioctylamine and mixtures thereof.

Aliphatic amines that can be prepared herein include octyldimethylamine, octylmonomethylamine, dioctylmethylamine, octylamine, decyldimethylamine, decylmonomethylamine, didecylmethylamine, decylamine, dodecyldimethylamine, dodecylmonomethylamine, didodecylmethylamine, didodecylamine, dodecylamine, 2-ethylhexyldimethylamine, oleyldimethylamine, tetradecyldimethylamine, tetradecylmonomethylamine, ditetradecylmethylamine, tetradecylamine, hexadecyldimethylamine and octadecyldimethylamine.

In one preferred embodiment of the present invention, the aminating agent is a secondary amine having the formula of (VI):

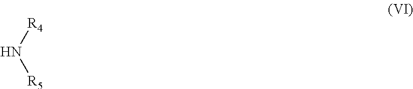

wherein R₄ and R₅, the same or different, are a linear or branched, saturated or unsaturated aliphatic group having from 1 to 24 carbon atoms, preferably from 1 to 18 carbon atoms, more preferably from 1 to 4 carbon atoms. Accordingly, the aliphatic amine that is formed has the formula (VII):

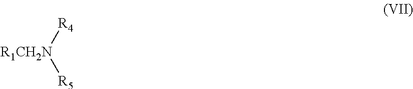

wherein R₁ is as defined in formula (I), R₄ and R₅ are as defined in formula (VI).

According to the present invention, the reaction of the aliphatic alcohol and the aminating agent is carried out in the presence of a copper containing catalyst. The catalyst of the present invention comprises from 68 wt % to 100 wt % of a copper oxide and from 0 wt % to 0.1 wt % of a metal co-catalyst, and optionally from 0 wt % to 32 wt % of a catalyst support, weight percentage is based on the total weight of the catalyst. Preferably, the catalyst comprises from 72 wt % to 100 wt % of a copper oxide and from 0 wt % to 0.1 wt % of a metal co-catalyst, and optionally from 0 wt % to 28 wt % of a catalyst support, weight percentage is based on the total weight of the catalyst. More preferably, the catalyst comprises from 75 wt % to 100 wt % of a copper oxide and from 0 wt % to 0.1 wt % of a metal co-catalyst, and optionally from 0 wt % to 25 wt % of a catalyst support, weight percentage is based on the total weight of the catalyst.

In some aspects, the catalyst comprises copper oxide as the sole catalytic metal, which means the catalyst does not contain any metal co-catalyst, such as Zn, Ni, Cr and alkaline metals (e.g. Ba and Mg). Accordingly, in one preferred embodiment of the present invention, the catalyst comprises from 68 wt % to 100 wt % of a copper oxide as the sole catalytic metal, and optionally from 0 wt % to 32 wt % of a catalyst support, weight percentage is based on the total weight of the catalyst. More preferably, the catalyst comprises from 72 wt % to 100 wt % of a copper oxide as the sole catalytic metal, and optionally from 0 wt % to 28 wt % of a catalyst support, weight percentage is based on the total weight of the catalyst. Even more preferably, the catalyst comprises from 75 wt % to 100 wt % of a copper oxide as the sole catalytic metal, and optionally from 0 wt % to 25 wt % of a catalyst support, weight percentage is based on the total weight of the catalyst.

The catalyst of the present invention preferably comprises a catalyst support. Accordingly, in a preferred embodiment of the present invention, the catalyst consists of from 68 wt % to 95 wt % of a copper oxide and from 5 wt % to 32 wt % of a catalyst support, weight percentage is based on the total weight of the catalyst. More preferably, the catalyst consists of from 72 wt % to 95 wt % of a copper oxide and from 5 wt % to 28 wt % of a catalyst support, weight percentage is based on the total weight of the catalyst. Even more preferably, the catalyst consists of from 75 wt % to 95 wt % of a copper oxide and from 5 wt % to 25 wt % of a catalyst support, weight percentage is based on the total weight of the catalyst.

Suitable catalyst support may be selected from alumina, such as Y-alumina, silica, magnesium aluminate, charcoal, kaolin and zeolite. Preferably, the catalyst support is silica.

The catalyst of the present invention can notably be prepared by co-precipitation method. The catalyst can be prepared by precipitating one or more of the components from solution. For example, copper hydroxide can be co-precipitated from a water solution by dissolving a water soluble salt of copper such as copper nitrate, in water, adjusting the pH thereof with a suitable base, to a pH of 7 to 12, resulting in the precipitation of the copper hydroxide. If desired, the materials of the catalyst support can be added into the mixture before the precipitation takes place. After filtering, the recovered copper hydroxide (optionally with the catalyst support) can be washed with water to obtain a high active catalyst. The obtained catalyst can be oven dried at a temperature of 80° C. to 200° C. for one to 48 hours. Alternatively the catalyst can be spray dried. Then the dried catalyst can be subject to calcination by using processes well known in the art.

In order to carry out the process of the invention, the reactant aliphatic alcohol and the reactant aminating agent are mixed according to a desired molar ratio. The reactant mixture is preferably mixed together with a flow of hydrogen. The mixture may be preheated to 200-400° C. Then the mixture may be continuously introduced into a reaction zone. The molar ratio of the aliphatic alcohol to the aminating agent may be in the range of from 1:1 to 1:2, preferably, in the range of from 1:1 to 1.5, more preferably, in the range of from 1:1 to 1:1.2. The molar ratio of the aliphatic alcohol to the hydrogen may be in the range of from 1:5 to 1:20, preferably in the range of from 1:10 to 1:18, more preferably in the range of from 1:10 to 1:15. Alternatively, an inert gas, such as nitrogen, can be added into the reactant mixture and introduced into the reaction zone as well.

Preferably, the reactant mixture is introduced into the reaction zone in vapour phase, at a liquid hourly space velocity (volume of liquid alcohol per volume of catalyst per hour) of from 0.05 to 5.0 kg of alcohol per kg of catalyst per hour, preferably from 0.1 to 2.0 kg of alcohol per kg of catalyst per hour, more preferably, from 0.5 to 1 kg of alcohol per kg of catalyst per hour.

The reaction is preferably carried out over a fixed bed wherein the catalyst according to the present invention is loaded. It is appreciated that the reaction may also be carried out in a stirring vessel which can be heated and which is provided with a device for the circulation of the reactant mixture.

The temperature of the reaction may be in the range of from 150° C. to 350° C., preferably, in the range of from 200° C. to 300° C., more preferably, in the range of from 200° C. to 250° C. The pressure in the reaction zone may be in the range of 0 to 5 barg, preferably in the range of 0 to 2 barg, more preferably, in the range of from 0 to 1 barg, even more preferably, in the range of from 0 to 0.5 barg.

According to the present invention, after the reaction has completed, the reaction product in the reaction zone may be subject to further steps such as distillation, condensation and recycling, by using procedures which are well known by a person skilled in the art, so as to recover the desired products. In one embodiment, the effluent in the reaction zone which containing the desired alkyl dimethylamine product is passed through a condenser to cool the reaction product to 30 to 150° C. Hydrogen, unreacted dimethylamine, water and small amount of monomethylamine and trimethylamine by products are removed overhead. The condensed liquid product is then sent to a distillation stage, wherein the desired alkyldimethylamine product is separated from heavier products, such as dialkyl methylamines.

It is appreciated that the process of the present invention may employ a single fixed bed reactor or multiple fixed bed reactors. In the latter case, for example, the process may employs two fixed bed reactors, wherein the reaction product in the first fixed bed reactor is introduced into the second fixed bed reactor together with a fresh stream of the aminating agent. The conditions in the second fixed bed reactor may be substantially same as those in the first fixed bed reactor as described above. Preferably, the reaction temperature in the second fixed bed reactor is 5-30° C. lower than that in the first fixed bed reactor.

### Examples

The following catalysts were employed for the present experiments:

TABLE 1

| | Catalytic metals | | | |
|---|---|---|---|---|
| | Copper | Chromium | Barium | |
| Catalyst | oxide | oxide | oxide | Catalyst support |
| No. 1 | 75 wt % | — | — | Silica (25 wt %) |
| No. 2 | 79 wt % | 21 wt % | — | — |
| No. 3 | 55 wt % | 24 wt % | 6 wt % | Silica (15 wt %) |

For the preparation of the No. 1 catalyst, Cu(NO₃)₂.3H₂O was dissolved in distilled water. Silica was also added into the solution. The solution was stirred and its pH was adjusted to 10 by slow addition of sodium hydroxide solution. A precipitate was formed which was recovered by filtering the solution through a medium porosity fritted glass filter funnel. The filter was washed with distilled water and extruded into shaped form. The extruded catalyst was subsequently dried and then calcined in air at ambient pressure and a temperature of 400° C. for about 24 hours.

For the preparation of the No. 2 catalyst, the process is same as that described for No. 1 catalyst expect that a mixture of soluble copper salt and soluble chromium salt was used instead of Cu(NO₃)₂.3H₂O and silica was not used for the preparation. For the preparation of the No. 3 catalyst, the procedure is same as described for No. 1 catalyst except that a mixture of soluble salt of copper, soluble salt of chromium and soluble salt of barium was used instead of Cu(NO₃)₂.3H₂O.

In order to investigate the catalytic behaviours of the above mentioned catalysts, the catalysts were loaded into a tubular fixed bed reactor (2 inch diameter and 1 meter length), respectively. Then, a mixture of dodecyl alcohol and dimethylamine, together with a flow of hydrogen gas were heated through a gasifier at 215° C. The molar ratio of the alcohol/dimethylamine/hydrogen was 1:1.2:14.7. Then the preheated mixture, which was in vapor phase, was introduced into the fixed bed reactor loaded with the catalyst, at a feeding rate of 0.5 kg of alcohol per kg of catalyst per hour. The reaction temperature in the fixed bed reactor was set at 215° C. and the pressure was set at 0.2 barg. The product stream from an outlet of the fixed bed reactor was cooled down to ambient temperature through a heat exchanger and samples of the product stream were collected for analysis. The samples collected were subject to gas chromatograph analysis. The components and the percentages thereof were measured and the results were shown in the table below:

TABLE 2

| | Catalyst | Catalyst | Catalyst |
|---|---|---|---|
| | No. 1 | No. 2 | No. 3 |
| Aldehyde (wt %) | 0.05 | 0.07 | 0.12 |
| dodecyl alcohol (wt %) | 7.5 | 13.93 | 22.97 |
| N′N-dimethyl dodecylamine (wt %) | 92.2 | 84.44 | 75.31 |
| N,N-didodecylmethylamine (wt %) | 0.16 | 1.50 | 1.54 |
| N-dodecylmethylamine (wt %) | 0.13 | 0.06 | 0.06 |
| Ester (wt %) | None | None | None |

As shown in Table 2, it has been found that catalyst comprising copper oxide as the sole catalytic metal lead to markedly higher percentage of desired tertiary amine product (N′N-dimethyl dodecylamine) in the product mixture and markedly higher conversion rate of dodecyl alcohol compared to catalysts comprising copper oxide and other catalytic metal(s). Furthermore, the presence of the catalyst support may also contribute to the enhanced efficiency and enhanced selectivity.

1. A process, comprising reacting an aliphatic alcohol of formula (I)
R₁CH₂OH (I),
wherein R₁ is a linear or branched, saturated or unsaturated aliphatic group having from 3 to 21 carbon atoms, with an aminating agent of formula (II) 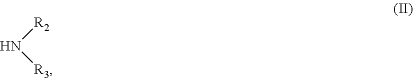
wherein R₂ and R₃, the same or different, are hydrogen or a linear or branched, saturated or unsaturated aliphatic group having from 1 to 24 carbon atoms, for obtaining an aliphatic amine of formula (III), (IV) or (V) 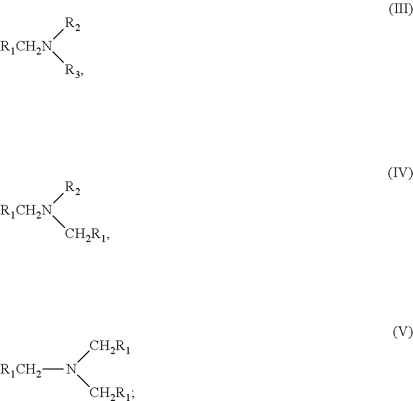
wherein the reaction is carried out in the presence of a catalyst comprising from 68 wt % to 100 wt % of a copper oxide and from 0 wt % to 0.1 wt % of a metal co-catalyst, and optionally from 0 wt % to 32 wt % of a catalyst support, weight percentage is based on the total weight of the catalyst.

2. The process according to claim 1, wherein the catalyst comprises from 75 wt % to 100 wt % of a copper oxide and from 0 wt % to 0.1 wt % of a metal co-catalyst, and optionally from 0 wt % to 25 wt % of a catalyst support, weight percentage is based on the total weight of the catalyst.

3. The process according to claim 1, wherein the catalyst comprises from 68 wt % to 95 wt % of a copper oxide as the sole catalytic metal, and optionally from 5 wt % to 32 wt % of a catalyst support, weight percentage is based on the total weight of the catalyst.

4. The process according to claim 1, wherein the catalyst comprises from 75 wt % to 100 wt % of a copper oxide as the sole catalytic metal, and optionally from 0 wt % to 25 wt % of a catalyst support, weight percentage is based on the total weight of the catalyst.

5. The process according to claim 1, wherein the catalyst consists of from 68 wt % to 95 wt % of a copper oxide and from 5 wt % to 32 wt % of a catalyst support, weight percentage is based on the total weight of the catalyst.

6. The process according to claim 1, wherein the catalyst consists of from 75 wt % to 95 wt % of a copper oxide and from 5 wt % to 25 wt % of a catalyst support, weight percentage is based on the total weight of the catalyst.

7. The process according to claim 1, wherein the catalyst support is silica.

8. The process according to claim 1, wherein the aminating agent has the formula (VI) 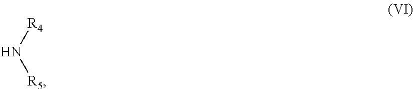
wherein R₄ and R₅, the same or different, are a linear or branched, saturated or unsaturated aliphatic group having from 1 to 24 carbon atoms, the aliphatic amine has the formula (VII): 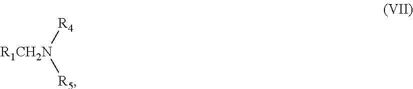
wherein R₁ is a linear or branched, saturated or unsaturated aliphatic group having from 3 to 21 carbon atoms, R₄ and R₅ are as defined in formula (VI).

9. The process according to claim 1, wherein the aliphatic alcohol and the aminating agent are mixed together with a flow of hydrogen and the mixture is continuously introduced into a reaction zone, wherein the molar ratio of the aliphatic alcohol/the aminating agent/the hydrogen is in the range of from 1:1:5 to 1:2:20.

10. The process according to claim 9, wherein the molar ratio of the aliphatic alcohol/the aminating agent/the hydrogen is in the range of from 1:1:5 to 1:1.2:15.

11. The process according to claim 1, wherein the reaction is carried out at a temperature of from 150° C. to 350° C.

12. The process according to claim 1, wherein the reaction is carried out at a temperature of from 200° C. to 250° C.

13. The process according to claim 1, wherein the reaction is carried out under a pressure of from 0 to 5 barg.

14. The process according to claim 1, wherein the reaction is carried out under a pressure of from 0 to 0.5 barg.
